(19) United States
(12) Patent Application Publication
(10) Pub. No.: US 2010/0198157 A1
(43) Pub. Date: Aug. 5, 2010

(54) Cannula and Delivery Device

(76) Inventors: Steffen Gyrn, Ringsted (DK); Orla Mathiasen, Soro (DK); Tenna M. Pedersen, Frederiksberg (DK)

Correspondence Address: BRINKS HOFER GILSON & LIONE, P.O. BOX 10395, CHICAGO, IL, 60610, US

(21) Appl. No.: 12/598,825

(22) PCT Filed: Dec. 6, 2007
(86) PCT No.: PCT/EP2007/063389

§ 371 Date: Apr. 12, 2010

Related U.S. Application Data
(60) Provisional application No. 60/928,025, filed on May 7, 2007.

(51) Int. Cl. A61M 5/142; A61M 5/14; A61B 17/34

(52) U.S. Cl. 604/151; 604/174; 604/164.08

(57) ABSTRACT

The present invention relates to a base part comprising a cannula device for use in delivery devices delivering medication to a patient under controlled conditions, and an inserter device for insertion of the cannula device in the base part. The base part comprises a receiving portion (7) for a cannula device (1), a portion (9) which can be placed on the skin of a patient and attachment parts (11) for a delivery part comprising at least a reservoir (13). The base part further has a connection transferring fluid from the reservoir (13) to the base part which connection can be provided at different angles relative to the base part (9).

The present invention relates to a base part comprising a cannula device for use in delivery devices delivering medication to a patient under controlled conditions, and an inserter device for insertion of the cannula device in the base part.

### PRIOR ART

Often delivery devices for intermittent or continuous administration of a therapeutical substance, such as insulin, are in form of a two-part device. Such a traditional delivery device comprises a base part having a cannula for subcutaneous insertion into a patient and comprising means for fastening of the base part to the patients skin, further the base part has means for closing of fluid access to the base part and it has means for opening of fluid access e.g. for receiving a connector cannula extending from a connector part and for bringing the connector cannula into fluid communication with the cannula of the base part. Often, the connector part is in fluid communication with a drug delivery device, e.g. an insulin pump.

Different kinds of delivery devices are described in WO 02/068014 A2, EP 0 956 879 A1, U.S. Pat. No. 5,522,803, US 2003/0225373 A1 and WO 03/026728 A1.

US 2003/0176852A1 discloses a delivery device in which a base part comprises a pivoting member, said base part comprising a cannula for insertion into a patient and pivoting member has an inner cavity with one receiving end adapted to receive an inserter needle or a connector cannula and two connecting ends (3161 and 320) for further connection with the cannula of the base part. During insertion the pivoting member is positioned orthogonal to the base part and an inserter needle penetrates a membrane in the receiving end and the needle passes through a canal and through the first connecting end into the cannula which then can be inserted. After insertion the needle is removed and the pivoting member is connected with a connector. The connector and the pivoting member are connected from the same direction as the connection between the pivoting member and the inserter. The pivoting member is then turned in order for the second connecting end to align with the cannula. This device has the drawback that it is very sensitive to movement of the pivoting member since a small movement will close of the delivery of drugs.

WO 02/094352 A2 discloses a delivery device having in the base part a construction that makes it possible to receive an insertion needle from one direction and a connector needle from a second direction. This design does not allow the patient to choose from which direction he/she wants to connect the connector with the base part.

In these prior art delivery devices the construction of the cannula and the means for providing fluid communication between the cannula and the cannula from the connector is unique for each set. Normally each infusion set also utilizes a specific set of guiding and/or locking means thus allowing only for a specific connector to engage with the base part.

WO 06/015600 A1 discloses a delivery device having a universal part having a cannula and means adapted to receive the cannula from the connector and fitting to most/all common infusion sets were available. This design allows for different types of connectors to be used with the same base part and visa versa, and it will also be possible to connect the connector from different angles.

The object of the present invention is to provide a cannula device which can be used as a component in different types of delivery devices and which is applied after a base part has been applied to the patient's skin.

According to the invention there is provided a cannula device for mounting in a base part comprising a housing and at least one membrane together defining at least one cavity, the cannula device further comprises a cannula mounted in the housing and being in fluid communication with the at least one cavity, which cannula device is provided with means for attaching the device to the base part on the proximal side of the device.

The advantage of such a cannula device is that it can be used as a standard component in delivery devices whether the delivery device has inclined or orthogonal insertion of the cannula. Thus this standard component can be mass produced and be used as a component in series of desired designs of the delivery devices. This results in lower manufacturing costs, a more flexible production line and a more flexible product. The positioning of the attaching means on the proximal side, i.e. the side turned towards the patient after mounting, of the device makes it easier to position the cannula device correctly by insertion as it is possible to cover or connect the sides of the cannula device with a handle or inserter device. Thus the attaching means will assure the attachment to the base part or receiving part while the side parts of the attaching means will assure the adaptation to the insertion device.

In another embodiment the cannula device for mounting in a base part comprises a housing and at least one membrane together defining at least one cavity, the cannula device further comprises a cannula mounted in the housing and being in fluid communication with the at least one cavity, where the cannula device is provided with means for attaching the cannula device unreleasably to the base part, i.e. to a specially adapted receiving portion of the base part.

The cannula device is normally a disposable device which is thrown away after use as the cannula is in contact with the patient's blood. If the base part to which the cannula device is attached also is a disposable device with approximately the same operating life it will not be necessary to be able to remove the device from the base part as both cannula device and base part will be removed and disposed of normally after having been used for a few days. When it is not possible to remove the cannula device from the base part it is not possible to have a used cannula device confused with a new sterile cannula device and it will also be evident that the receiving portion of the base part in which a cannula device is locked is not suitable for use.

If the base part is of a type which can be attached to the patient for a longer period, it might be possible to insert a new cannula device at a different position while the used cannula device is removed from the subcutaneous position e.g. by removing the cannula device together with a receiving portion or a part of a receiving portion to which it might be permanently attached.

Both embodiments have the advantage that it is possible for the user first to carefully position the base part, and after having positioned the base part properly, then the user can concentrate on injecting the cannula device.

In one embodiment the means for attaching the device to the base part comprise mechanical features cooperating with corresponding means on the base part, e.g. the means for attaching the device to the base part comprise parts extending from a proximal surface of the cannula device which parts can pivot and thereby temporarily reduce the diameter in at least one position or the means for attaching the device to the base part can comprise an adhesive surface on a proximal surface of the cannula device adhering to a corresponding surface of the base part.

In one embodiment the cannula device is provided with guiding means corresponding to an inserter device which guiding means secure a well-defined motion of the cannula device when being moved towards the base part by the inserter device.

In one embodiment the cannula device is inserted with an inserter device provided with a covering part covering the full length of the cannula device.

In one embodiment the part of the body of the cannula device having the largest diameter is rotational-symmetrical around a central axis.

In another embodiment the part of the body of the cannula device having the largest diameter has angled sides e.g. providing a triangular or quadrangular profile when cut-through. When having angled sides the profile of the cannula device can be used to define the correct insertion position.

In one embodiment the cannula device comprise a body showing a smooth outer surface and having an inner cavity, the inner cavity is at the distal end covered with a wall such as a membrane or a septum which can be penetrated by a needle such as a connector needle or a syringe and at the proximal end of the inner cavity a cannula is embedded, the outer proximal surface of the body, i.e. a surface of the body facing the receiving portion during injection of the cannula device, is provided with means for unreleasably attaching the device to a receiving portion. The smooth outer surface can e.g. have a round or oval circumference and the wall covering the distal end of the inner cavity can be penetrated either by a pointy or by a blunt needle which ever might be preferred.

In one embodiment the unreleasable attachment between the receiving portion and the cannula device is formed automatically, that is without the need to take any action in order to form the unreleasable attachment, as the cannula device is pushed against the receiving portion.

According to another aspect of the present invention, a delivery device is provided. The delivery device includes a base part provided with a receiving portion for a cannula device where the receiving section has guiding means for an inserter device which inserter device holds the cannula device before insertion, i.e. the receiving portion has no guiding means for the cannula device or at least the receiving portion does not need guiding means for the cannula device as the guiding means for the inserter device might provide sufficient guidance for correct positioning of the cannula device.

In one embodiment the cannula device corresponds to an internal opening in a part of the inserter and the cannula device is provided with means for attaching the device to the base part on the proximal side of the body of the cannula device.

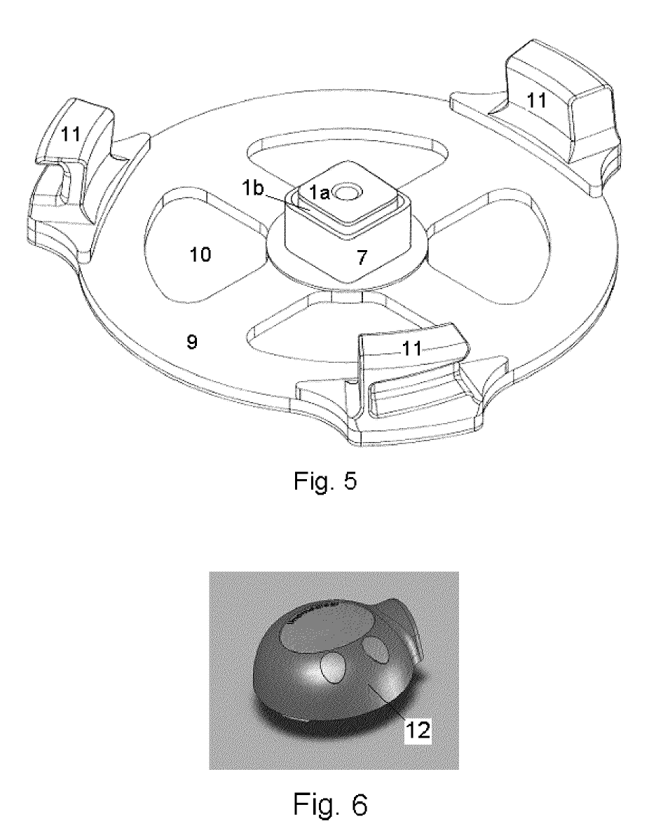

According to another aspect of the invention another base part is provided. This base part comprises a receiving portion for a cannula device, a portion which can be placed on the skin of a patient and attachment parts for a delivery part comprising at least a reservoir. The delivery part which can be connected to this base part has more than one position relative to the attachment parts. In a first position parts of the delivery part corresponding to the attachment parts of the base part is/are brought into contact with the attachments parts, after contact is made the parts of the delivery part corresponding to the attachment parts of the base part slides along a track or a surface towards a second position where a fluid connection between the delivery part and the cannula device) is formed. The tracks or surface can be a continuous opening in an upright wall as shown e.g. in FIG. 5 or 8, or it can be the outer/upper of a protruding surface leading towards the receiving portion. If the tracks or surface is a continuous opening in an upright wall, then the corresponding parts of the delivery part could be protruding parts engaging the openings. If the tracks or surface is a band then the corresponding parts of the delivery part are corresponding sliding parts.

The cannula device will be described in further detail with reference to the figures:

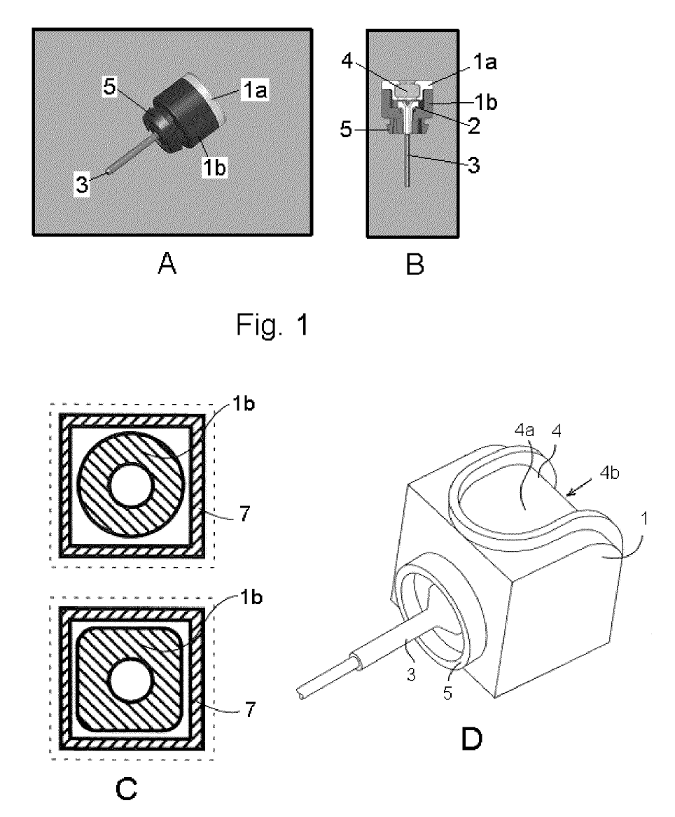

FIG. 1A is a view of an embodiment of the cannula device of the present invention, B is a cut-through view of the same cannula device as shown in A, C is a cut-through view along line A-A of FIG. 1B of a cannula device having a square profile, D shows a cannula device having a square profile and to access positions from different angles;

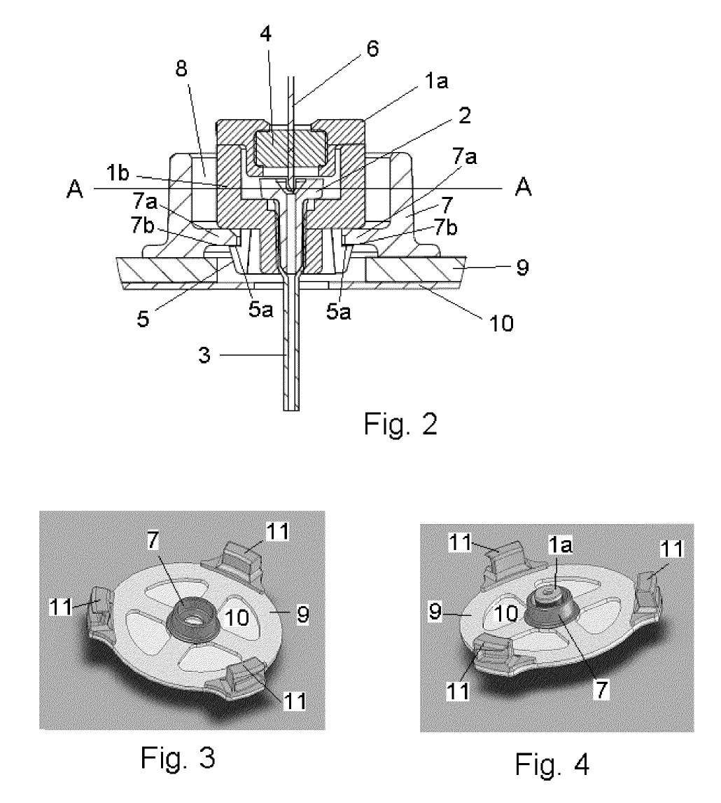

FIG. 2 is a cut-through view of an embodiment of the cannula device of the present invention placed in a receiving portion of a delivery device;

FIG. 3 is a view from above of a base part with a receiving portion in the center;

FIG. 4 is a view from above of a base part with a receiving portion in the center and a cannula device with a round profile positioned in the receiving portion;

FIG. 5 is a view from above of a base part with a receiving portion in the center and a cannula device with a square profile positioned in the receiving section:

FIG. 6 is a view from above of a base part on which a top comprising a reservoir and means for transporting the content of the reservoir to the patient has been mounted;

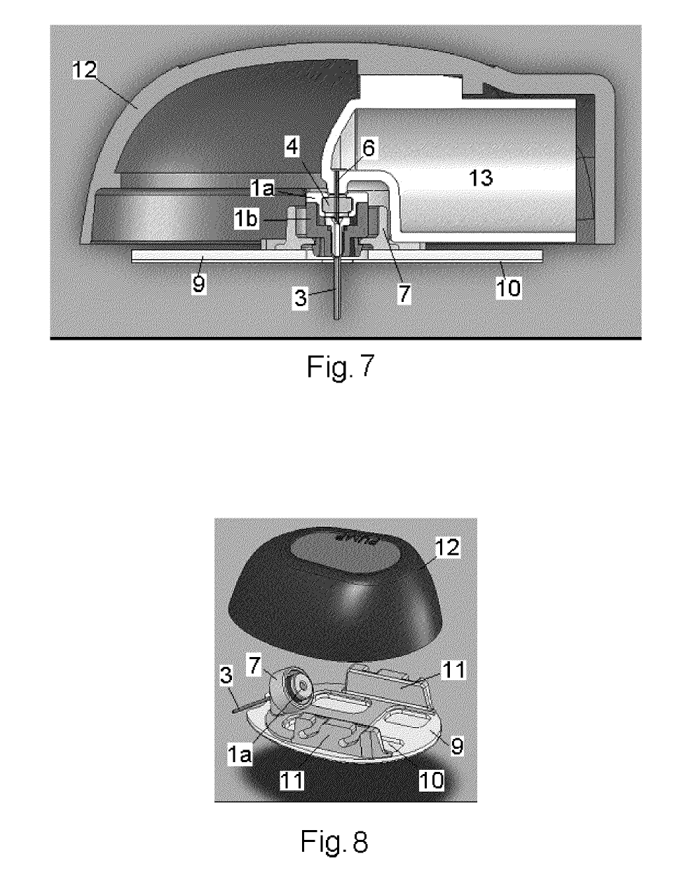

FIG. 7 is a cut-through view of the delivery device shown in FIG. 5;

FIG. 8 is a cut-through view of another embodiment of a delivery device according to the invention;

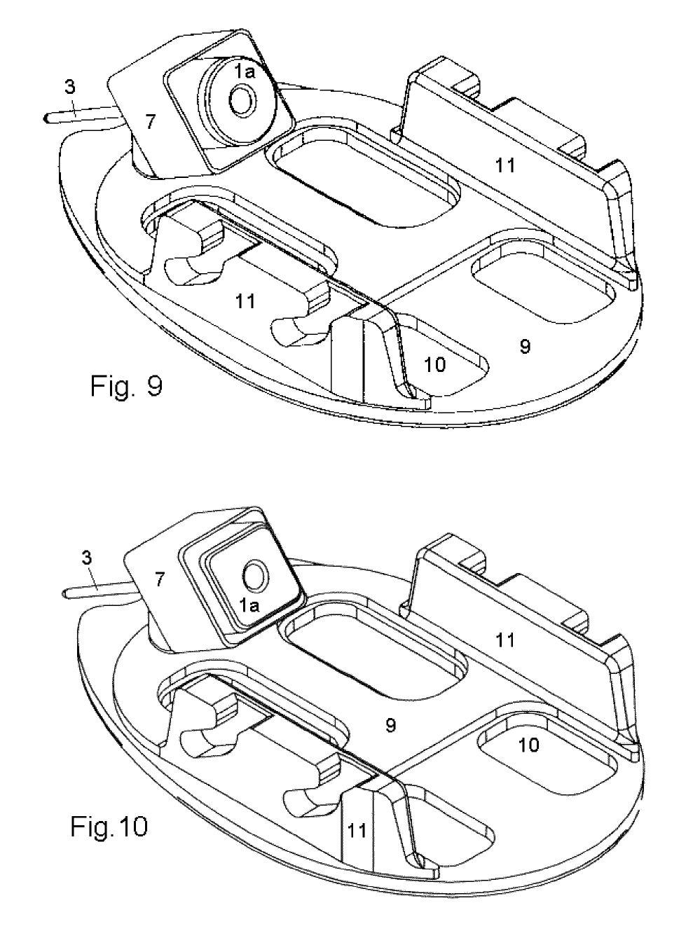

FIG. 9 shows a base part of an embodiment having a loose fit square receiving section for a round cannula device;

FIG. 10 shows a base part of an embodiment having a close fit square receiving section for a square cannula device;

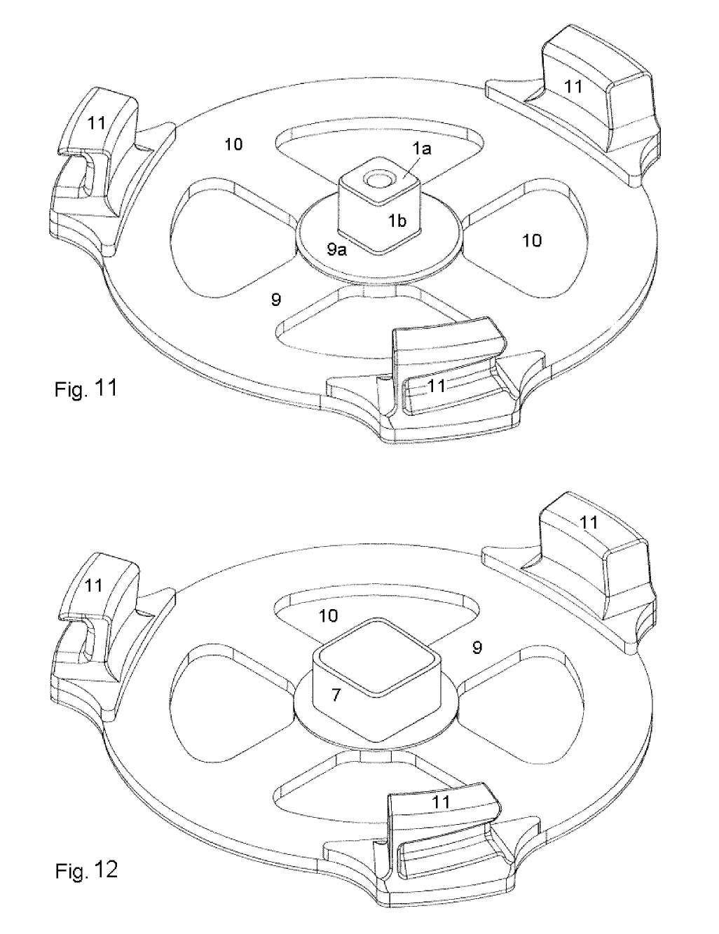

FIG. 11 shows a base part of an embodiment having a receiving section without upright walls for any cannula device;

FIG. 12 shows a base part of an embodiment having a square receiving section for a not shown cannula device;

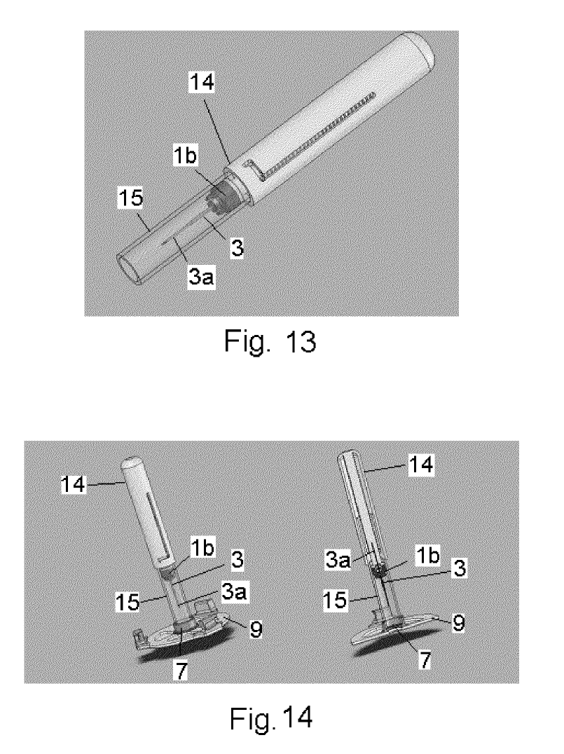

FIG. 13 is a side view showing the cannula device mounted in an inserter prepared for injection;

FIG. 14 shows two views of the same embodiment of a cannula according to the invention mounted in an inserter which inserter is joined to a receiving portion of a base part;

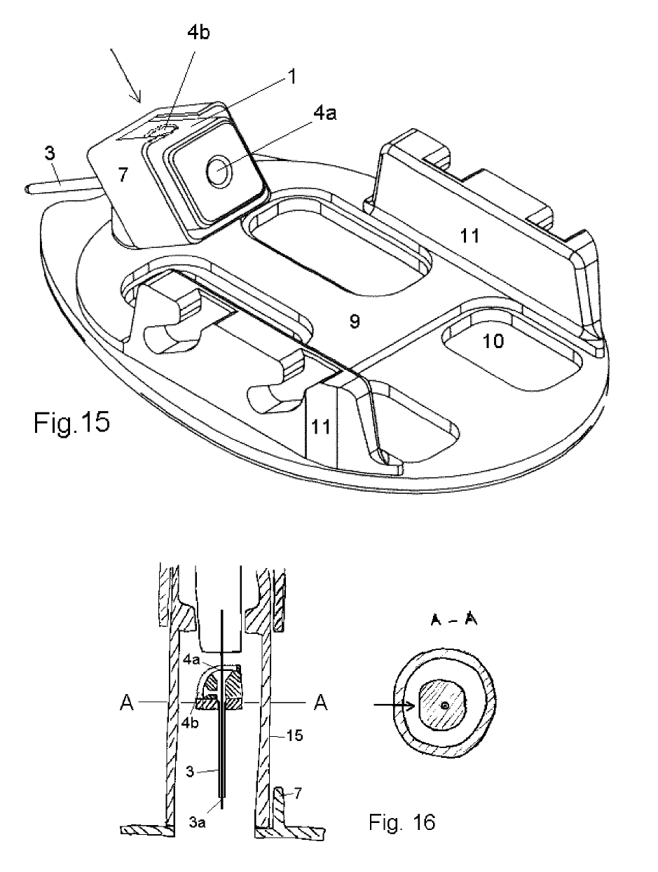

FIG. 15 shows a base part having a close fit square receiving section for a square cannula device with two access openings;

FIG. 16 shows a cannula device with two access openings placed in an inserter positioned in contact with a base part:

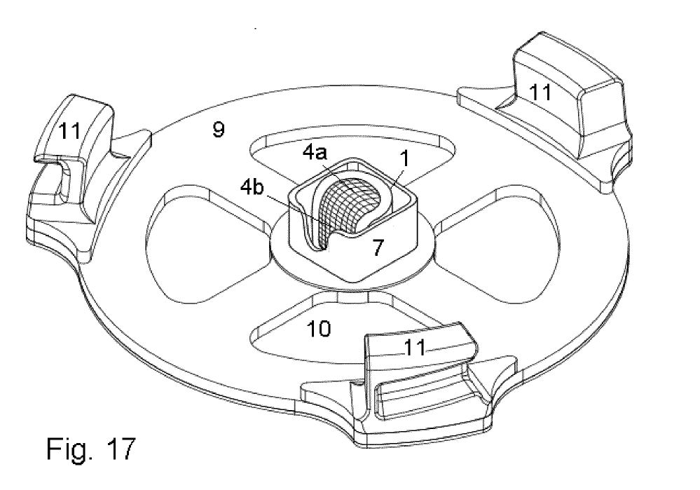

FIG. 17 shows a base part having a centrally positioned receiving portion adapted to a cannula device having to access openings.

### DETAILED DESCRIPTION OF THE INVENTION

FIGS. 1A and B show a first embodiment of the present invention. In this embodiment, the cannula device includes a housing 1a, 1b and a wall in the form of e.g. a membrane 4 which together define an inner cavity adapted to receive a piercing member 6 extending from e.g. a connector or a syringe. The housing 1a, 1b is normally made of a relatively hard molded plastic material.

The lower part 1b can be constructed of a cylindrical upper part where the inner surface forms the walls of the inner cavity and the outer surface is smooth and without protrusions, and of a cylindrical lower part with a smaller diameter where the inner surface forms an opening which supports a cannula 3 and the outer surface comprise means 5 for attaching the cannula device unreleasably to a base part 9.

The cannula device can also be constructed with an angular profile e.g. a quadrangular profile as shown in FIG. 1C. This figure shows two embodiments of the cannula device: a device having a round profile (upper) and a device having a square profile (lower). This profile shows when the cannula device is seen from above along the line A-A shown in FIG. 2. Whether the profile of the cannula device is round or angular it might have a loose fit or a close fit in the receiving portion where a loose fit and transporting means for transferring fluid from the reservoir normally indicates that the receiving section is provided with guiding means for the inserter and the cannula device is placed in correct position when the inserter is placed according to the guiding means and when the inserter is removed from the receiving section an empty space corresponding to the walls of the inserter can appear around the cannula device. A close fit means that the receiving section of the base part is provided with a room closely corresponding to the form or the profile of the cannula device and the cannula device is positioned inside the room, e.g. by an inserter, which room has walls closely corresponding to the outer walls of the cannula device.

The cannula device shown in FIG. 1D has two access openings covered with one membrane 4, in another (not shown) embodiment each access opening could be covered with separate non-connected membranes at different surface positions (e.g. respectively 4a and 4b). Such a cannula device can be fed with medication from two different angles via the surface 4a or the surface 4b. Such a device provides the possibility of having an extra access for medication if a corresponding opening is provided in the receiving portion 7 or alternatively the cannula device could be a standard device for two different types of base parts 9 provided with different openings in the receiving portion 7.

The cannula is made of a soft inert material and in this embodiment the cannula 3 is attached to the housing 1b by pushing a fastening part 2 made of a more rigid material than the cannula 3 into the opening of the cylindrical lower part after positioning the cannula 3 in the opening. As the fastening part 2 is pushed into the opening the cannula 3 will be squeezed against the walls of the opening and this pressure will keep the cannula 3 in a correct position.

The means 5 for attaching the cannula device unreleasably to the base part 9 is in this embodiment constructed as several hooks 5; these hooks 5 can pivot around the position where they are attached to the housing 1a, in this embodiment the attachments for the hooks 5 are of a flexible material i.e. the hooks can be pushed inwards when the hooks 5 pass an area of reduced diameter. Each hook 5 is provided with an upper surface 5a parallel to a contact surface 7b of a receiving portion 7 of the base part 9. Each hook 5 is also provided with an inclined surface 5b which inclined surface during insertion of the cannula device is in contact with a protruding part 7a of the receiving portion 7. When the cannula device is pushed down into the receiving portion 7 the hooks 5 are pushed inward against the lower cylindrical part of the housing 1b and as the hooks 5 in this position are biased, the hooks 5 will return to their original position when the inclined surface 5b of the hooks 5 has fully passed the protruding part 7a of the receiving portion 7. When the hooks 5 return to their original position the upper surface 5a of the hooks will be in touch with the contact surfaces 7b of the receiving portion 7 and the cannula device will be locked in this position as neither the cannula device nor the receiving portion 7 are provided with means to push the hooks 5 inward against the lower cylindrical part of the housing 1b.

According to another not shown embodiment either the upper side of the protruding parts 7a or the lower side of the housing 1a is provided with an adhesive which adhesive then works as unreleasably attaching means when the cannula device is pushed into position in the receiving portion 7.

FIG. 2 shows the same embodiment of the cannula device as in FIG. 1, where the cannula device is positioned in the receiving portion 7. The receiving portion 7 is provided with essentially vertically positioned walls covering the side section of the cannula device and a bottom part formed by the protruding parts 7a on which the cannula device rests when locked in the receiving portion 7. The essentially vertical walls which encircle or surround the cylindrical space 8 around the cannula device can create a guiding mean for an inserter. The cannula device is in this embodiment fully covered by a lower cylindrical part of the inserter and when the user wants to inject the cannula device, the cylindrical lower part of the inserter is placed in the space 8 formed by the receiving portion 7 and then the cannula device is pushed in position by a plunger being moved forward inside the cylindrical lower part of the inserter.

The receiving portion 7 is attached unreleasably to the base part 9 which base part 9 is fastened to the skin of a patient e.g. with a mounting pad 10.

FIG. 3 shows an upper view of a base part 9 provided with a receiving portion 7 and with attachment parts 11 for a delivery part. A delivery part might comprise both transporting means and a reservoir for medication but at least the reservoir. The transporting means normally have the form of a pump which provides for the controlled transport of medication from the reservoir to the patient. The control can be predefined in relation to the patient before the device is positioned on the patient e.g. the device can be programmed to deliver a constant amount prime unit, to deliver a certain dose at defined intervals or the delivering can be defined continuously by administering an amount or dose defined as a result of sensor measurements monitoring one or more components in the patients blood.

The base part 9 will normally be fastened to the patient by an adhesive surface or surface parts provided at the proximal side of the base part 9 or by a separate adhesive part or layer, but any kind of mounting which will make the base part stick to the patient without allowing the device to move can be used. A separate adhesive part or layer can be fastened to the base part 9 by glue, Velcro, molding or the like.

PCT applications PCT/DK2006/000737 and PCT application PCT/DK2006/000472 both filed on 22 Dec. 2006 relates to delivery devices of the described type having delivery parts comprising both reservoir and transporting means placed in joined relation to the base part. The delivery devices having base parts and delivery parts as shown and described in these two PCT applications are hereby incorporated by reference in the present application.

FIG. 4 shows an upper view of the same base part 9 as shown in FIG. 3 also provided with a receiving portion 7 and with attachment parts 11 for a delivery part but in FIG. 4 a cannula device has been positioned in the receiving portion 7.

FIG. 5 shows an upper view of another embodiment of a base part 9. FIG. 5 shows a centrally placed receiver 7 having a square profile in which a cannula device 1 having a square profile is placed. The receiving section 7 of this base part 9 has a square inner room and a cannula device 1a having an outer square profile is placed in the receiving section 7.

FIG. 6 shows an upper view of a base part 9 as shown in FIG. 3, 4 or 5 but in FIG. 6 the base part 9 has been provided with a cover 12 in which a delivery part comprising a reservoir 13 for medication and means for transporting of the medication from the reservoir to the patient are embedded.

FIG. 7 shows a cut-through view of the device shown in FIG. 6. FIG. 7 shows a receiving portion 7 positioned on a base part 9 which base part 9 has an underlying mounting pad 10. A cannula device has been inserted in the receiving portion 7 and the cannula 3 of the cannula device is inserted subcutaneously in a patient. The cannula device and the receiving portion 7 could be either square or round. The cover 12 is mounted releasably on the base part 9, and a connector needle 6 forms a fluid connection between the reservoir 13 which is attached to the inside of the cover 12 and the cannula device by penetrating the septum 4 of the cannula device. The transporting means are not shown.

The delivery device of FIG. 7 can be mounted on the patient through the following steps:

I. A sterile base part 9 is unpacked and secured to the skin of the patient.

II. A sterile single-use inserter including a cannula device is unpacked or a sterile part comprising an injection needle combined with a cannula device is unpacked an applied to a multiple-use inserter, the proximal end of the inserter is placed in the guiding means 8 of the receiving portion 7 and the cannula device is inserted, i.e. the cannula 3 is injected subcutaneously.

III. A delivery part comprising a cover 12, a reservoir 13 and means for transporting the content of the reservoir to the patient is fastened to the base part 9, and when the cover 12 is fastened to the base part 9 the connector needle 6 penetrates the septum 4 of the cannula device and then the delivery device is ready to work.

FIG. 8 shows another embodiment of a delivery device. In this embodiment the receiving portion 7 is positioned at the edge of the base part 9 and the cannula device having a cylindrical body 1b is not inserted perpendicular to the patient's skin but in an angle of approximately 30°. The reference numbers refers to similar parts as in FIGS. 6 and 7.

FIGS. 9 and 10 shows other embodiments of a delivery device. The embodiment of FIG. 9 is provided with a cylindrical cannula device (round profile) placed in a square or rectangular receiving portion 7. This embodiment provides a loose fit for the cannula device. The embodiment of FIG. 10 is provided with a square or rectangular cannula device placed in a square or rectangular receiving portion 7. This embodiment also provides a loose fit for the cannula device where the upright walls of the receiving portion 7 can provide the guiding means for an inserter.

In FIG. 9 the peripheral placed receiver has a square profile in which a cannula device 1 having a round profile is placed. In order to position this cannula device 1 correctly in the base part 9 an injection device having a square outer and a round inner profile is needed or at least an inserter which have parts or surfaces adapted to fit into an outer square space formed by the receiver and have parts or surfaces which can provide a space in which a round cannula holding device can slide.

In FIG. 10 the peripheral placed receiver has a square profile in which a cannula device 1 having a square profile is placed. In order to position this cannula device 1 correctly in the base part 9 an injection device having a square outer and a square inner profile is needed or at least an inserter which have parts or surfaces adapted to fit into an outer square space formed by the receiver and have parts or surfaces which can provide a space in which a square cannula holding device can slide.

FIG. 11 shows a centrally placed receiver without upright walls guiding the inserter into position. Instead the slightly raised circumference of the central plate 9a of the base part 9 corresponding to a part of the proximal end of the inserter indicates the correct position of the inserter during insertion of the cannula device 1.

FIG. 12 shows a base part 9 having a centrally placed receiver having upright walls which walls provide the receiver 7 with a square profile. The base part 9 is shown before the cannula device 1 inserted.

FIGS. 13 and 14 illustrates an inserter which can be used when inserting the cannula device in a base part of a delivery device with a receiving portion 7 shown in FIG. 3-10. Such an inserter should have outer walls providing a profile corresponding to a part e.g. the walls of the receiving portion 7 and inner walls providing a profile corresponding to the cannula device in question in order for the inserter to guide the cannula device into the correct position.

In order for the inserter 14 to interact properly with the receiving portion 7 of the base part 9 it is desirable that the inserter 14 is provided with guiding means 15 which extents beyond the end of the injection needle of the inserter. The guiding means 15 can have a triple purpose as they can serve 1) to keep the injection needle out of sight of the user before, during and after injection of the cannula device, 2) to protect the environment from the injection needle, and 3) to assure safe and precise injection of the cannula device into the receiving portion 7.

In this embodiment the guiding means 15 of the inserter has a form which corresponds to the shape of the guiding means 8 in the receiving portion 7, e.g. of a cylindrical or rectangular tube.

A detailed description of the specific inserter shown in FIGS. 8 and 9 and a description of how this inserter functions can be found in DK application PA200601028 filed on 2 Aug. 2006. This inserter is hereby incorporated by reference.

FIG. 15 shows an embodiment of a delivery device similar to the embodiments shown in FIGS. 9 and 10 but in the embodiment of FIG. 15 the receiver portion 7 has been changed in order to make it possible to inject liquid from to different angles. The cannula device has as shown in FIG. 1D two access openings but in the cannula device of FIG. 15 each opening is covered by a separate membrane 4b and 4a. The opening covered by the first membrane 4a can e.g. be used when applying the cover 12 inside which a fluid connection is formed to a reservoir 13 while the second access covered by the second membrane 4b e.g. can be used when another medication or a higher dose than the one flowing through the fluid connection from the reservoir 13, has to be injected. In this situation the cover 12 could be provided with a corresponding opening which would make it possible to inject extra or other kinds of medication without removing the cover.

FIG. 16 shows an embodiment of a cannula device intended for a base part 9 having a centrally positioned receiving portion 7. The cannula device 1 is placed in an inserter, it is held in position inside the inserter by the friction between the insertion needle 3a which is unreleasably connected to the inserter and the cannula 3 of the cannula device. That the insertion needle 3 is unreleasably connected means that it can be molded into the plunger of the inserter. The cannula device of FIG. 16 has two access openings, the insertion needle is placed through the first access opening covered by the membrane at position 4a and the second access opening is placed perpendicular to the first access opening and covered by the membrane at position 4b. When the cannula device is inserted into the base part 9 it will be possible to access both openings as the receiving portion 7 has been provided with an opening corresponding to the second opening of the cannula device.

FIG. 17 shows a base part 9 which can be used in combination with the cannula device of FIG. 16 i.e. the base part 9 provides several access openings for the delivery part. In FIG. 17 one access is provided in a direction approximately perpendicular to the upper surface of the base part 9, according to the shown embodiment approximately would mean ±10° to perpendicular of the upper surface of the base part 9. The possible insertion angle is defined both by the position of the membrane but also by the angle of the walls of the inner cavity in the cannula device 1 therefore the connector needle 6 of the delivery part does not need to be exactly perpendicular to the upper surface of the base part 9. The second access opening is provided in a direction approximately parallel to the upper surface of the base part 9 i.e. the direction of the connection needle need not be exactly parallel to the upper surface of the base part 9 but could deviate ±10° from parallel or as much as the walls of the inner cavity of the cannula device allow. In this embodiment the first connection angle, i.e. the connection through the first access opening, and the second connection angle, i.e. the connection through the second access opening, deviate from each other by around 90°, i.e. between 70° and 110°.

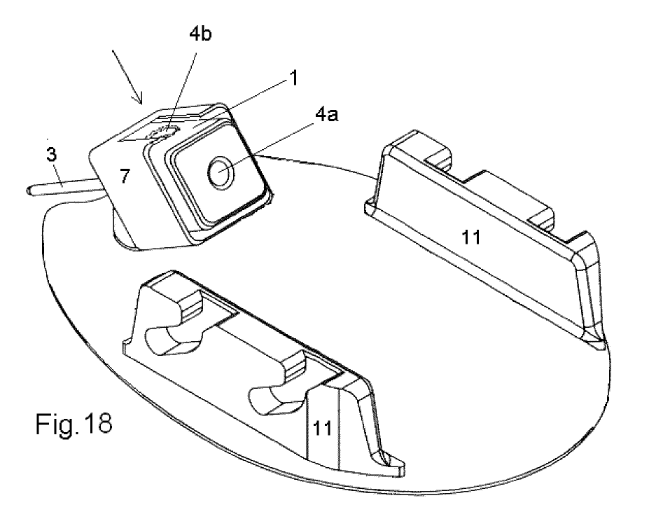

FIG. 18 shows a base part 9 of similar type as the one shown in FIG. 17 but in this embodiment the base part 9 is of the form and size of a credit card without a separate mounting pad. The base part 9 can be made by molding without a separate mounting pad and provided with an adhesive on the proximal side which secures the device to the user after mounting.

The openings of the attachment parts 11 are inclined towards the receiving portion in order for the delivery part to approach the receiving portion 7 in a correct angle. Actually the surfaces need not be inclined; they can have any form or direction directing the delivery part to connect with the base part 9 or the cannula part 1 in a desired way. When joining the delivery part to the base part 9, the delivery part is first positioned on the attachments parts 11 in a position away from the receiving portion and then the delivery part slides along the attachment parts 11 until the final position is reached and a fluid connection is made between the delivery part and the cannula part 1. According to a similar embodiment the attachment parts 11 could comprise a single longish central part instead of two separate parts placed near the sides of the base part 9.

A cannula device according to the present invention can appropriately be used in relation with treatment of diabetes or in relation with deliverance of other drugs where the cannula device is connected to a reservoir and a pump unit or the cannula device can be a part of a gate way system where syringes can be used to feed one or more different drugs to the patient.

A cannula device according to the present invention can also consist of a sensor or a probe which have to have a part positioned subcutaneously in contact with the blood stream of the patient i.e. in order to meter the glucose content of the patient's blood.

1. A base part comprising a receiving portion (7) for a cannula device (1), a portion (9) which can be placed on the skin of a patient and attachment parts (11) for a delivery part comprising at least a reservoir (13), characterized in that a connection transferring fluid from the reservoir (13) to the base part can be provided at different angles relative to the base part (9).

2. A base part according to claim 1, characterized in that the delivery device further comprises transporting means for transferring fluid from the reservoir (13) to the patient.

3. A base part according to claim 1, characterized in that the transferring means are a pump.

4. A base part according to claim characterized in that at least two connection angles deviates from each other by at least 40°, preferably by at least 60°.

5. A base part according to any of claims 1-2, characterized in that the cannula device (1) is provided with more than one access opening to an inner cavity.

6. A base part according to any of claims 1-5 characterized in that the cannula device is provided with means (5) for attaching the device to the base part (9) on the proximal side of the device.

7. A base part according to claim 6 characterized in that the means (5) for attaching the device to the base part (9) comprise mechanical features cooperating with corresponding means (7a) on the base part (9).

8. A base part according to claim 6 characterized in that the means for attaching the device to the base part (9) comprise parts (5) extending from a proximal surface of the cannula device which parts (5) can pivot and thereby temporarily reduce the diameter formed by the edges of the parts (5) in at least one position.

9. A base part according to claim 6 characterized in that the means (5) for attaching the device to the base part (9) comprise an adhesive surface on a proximal surface of the cannula device adhering to a corresponding surface of the base part (9).

10. A base part according to claim 1 characterized in that the cannula device is provided with guiding means corresponding to an inserter device (14) which guiding means secure a well-defined motion of the cannula device when being moved towards the base part (9).

11. A base part according to claim 10 characterized in that the cannula device is inserted with an inserter device (14) provided with a covering part (15) covering the full length of the cannula device.

12. A base part according to any preceding claim characterized in that the device comprise a body (1b) showing a smooth outer surface and having an inner cavity, the inner cavity is at the distal end covered with a wall (4) which can be penetrated by a needle (6) and at the proximal end of the inner cavity a cannula (3) is embedded, the outer proximal surface of the body (1b) is provided with means (5) for unreleasably attaching the device to a receiving portion (7).

13. A base part according to any preceding claim characterized in that the smooth outer surface of the cannula device (1) has a round, angular e.g. rectangular or oval circumference.

14. A base part according to any preceding claim characterized in that the wall (4, 4a, 4b) covering the access openings of the inner cavity can be penetrated by a pointy or blunt needle (6).

15. A base part according to claim 6 characterized in that an unreleasable attachment between the receiving portion (7) and the cannula device is formed automatically as the cannula device is pushed against the receiving portion (7).

16. A base part comprising a receiving portion (7) for a cannula device (1), a portion (9) which can be placed on the skin of a patient and attachment parts (11) for a delivery part comprising at least a reservoir (13), characterized in that the delivery part has more than one position relative to the attachment parts and in a first position parts of the delivery part corresponding to the attachment parts (11) of the base part (9) is/are brought into contact with the attachments parts (11) and then the parts of the delivery part corresponding to the attachment parts (11, 11a) of the base part (9) slides along a track or a surface towards a second position where a fluid connection between the delivery part and the cannula device (1) is formed.

17. An inserter device for insertion of a cannula device in a base part according to claims 1-16, said device comprising a first insertion part and a second insertion part, and an injection needle where
the second insertion part is connected to the injection needle and the injection needle is releasably combined with the cannula of the cannula device,
the first insertion part covers the injection needle in a non-activated position,
the first insertion part engages with the second insertion part, and said first insertion part is provided with guiding means interacting with corresponding guiding means of the second insertion part for guiding a slidable movement of the first and second insertion parts in relation to each other, and
the guiding means of the first and the second insertion part allows the injection needle to project beyond the first insertion part when the insertion device is activated.

18. An inserter device according to claim 18, where the cannula device has a retracted position inside the first insertion device and a forward position inside the first insertion device, and in the forward position the cannula (3) of the cannula device extends beyond the open proximal end of the first insertion device.

19. An inserter device according to claim 17, where the first insertion part is provided with guiding means which means can be combined with means of a base part (9) being secured to the patient in order to create a well-defined insertion point and angle.
